(19) United States
(12) Patent Application Publication
(10) Pub. No.: US 2022/0151824 A1
(43) Pub. Date: May 19, 2022

(54) Foot Cooler

(71) Applicants: Nenad Yashruti, Port Orchard, WA (US); Toni Paquette, Port Orchard, WA (US)

(72) Inventors: Nenad Yashruti, Port Orchard, WA (US); Toni Paquette, Port Orchard, WA (US)

(21) Appl. No.: 17/520,708

(22) Filed: Nov. 7, 2021

Related U.S. Application Data
(60) Provisional application No. 63/113,923, filed on Nov. 15, 2020.

(51) Int. Cl. A61F 7/02; A61F 7/00

(52) U.S. Cl. CPC A61F 7/02; A61F 7/0085; A61F 2007/0056; A61F 2007/0045; A61F 2007/0093; A61F 2007/0219; A61F 2007/0078; A61F 2007/0094; A61F 2007/0233

(57) ABSTRACT

A therapeutic sleeve and techniques for using it are provided. A therapeutic sleeve may house a conduit carrying a constant stream of a chilled or heated working fluid throughout the sleeve. A heating element may evenly distribute heat throughout the sleeve. The sleeve may provide a precise temperature-controlled pocket that significantly improves treatment of pain or discomfort in the user's feet, head, or other body parts. A software application may enable a user to set or adjust a temperature, set timers, record sleep patterns, and view analytics or other data.

### FIELD

This disclosure relates generally to a therapeutic sleeve.

### BACKGROUND

Many people suffer from conditions that manifest in pain or discomfort. Diabetes or peripheral neuropathy, for example, can reduce nerve function or restrict blood flow, causing acute or chronic pain in the limbs or extremities. The distance of a body part from the heart can also contribute to poor blood circulation or thermal discomfort. Medications to treat such conditions can have serious side effects, including addiction. Cryotherapy devices and other treatment methods on the market provide inconsistent relief and are extremely unsafe for prolonged use or during sleep.

### SUMMARY

The following presents a simplified summary of the disclosure to provide a basic understanding to the reader. This summary is not an extensive overview of the disclosure, nor does it identify key or critical elements of the claimed subject matter or define its scope. Its sole purpose is to present some concepts disclosed in a simplified form as a precursor to the more detailed description that is later presented.

Disclosed, among other things, is a therapeutic sleeve and techniques for its use. In one implementation, the therapeutic sleeve may comprise a thermally insulating material having a plurality of channels disposed within the therapeutic sleeve. The channels may receive one or more conduits operable to carry a constant stream of chilled or heated working fluid throughout the therapeutic sleeve. The conduits may attach to a cooling or heating plant, which may chill or heat the working fluid to a precise temperature for an exact duration, based on settings input by the user. In one implementation, channels may receive a heating element, such as a coil or ribbon resistance wire, to distribute heat throughout the sleeve. A thermal storage medium, for example, a gel, silicone, water, or another thermally conductive substance, may be disposed around the conduits or heating element to facilitate an even transfer of heat throughout the therapeutic sleeve.

In one implementation, the therapeutic sleeve may be integrated with a sheet, a garment, or another apparatus configured to improve accessibility or convenience to the user. For example, the therapeutic sleeve may be integrated with a bedsheet for any size bed, a hospital bed, a stretcher, a gurney, a mobile unit, or the like, such that the sleeve may receive the user's feet at or near the foot of the bed. The therapeutic sleeve may include straps, magnets, snaps, buttons, clips, adhesives, hook-and-loop fasteners, or another attachment means to secure to a bed, other furniture, or another object.

The therapeutic sleeve may operate with a software application that enables the user to input a desired temperature and duration, set a timer, track sleep patterns, record other data, or view analytics, among many possible functions, using a control panel, a remote control, or a mobile device, for example. In another implementation, the therapeutic sleeve may use battery-powered components, for example, a battery-powered cooling or heating plant or a battery-powered cooling or heating element, for ease of use and transportability.

The therapeutic sleeve may provide significant and much-needed improvements over conventional treatment methods and devices. Among its many benefits, the therapeutic sleeve's precise temperature controls may enable safer heating or cooling for prolonged use, even during sleep, mitigating or eliminating risks of burns, frostbites, skin irritation, or other safety hazards inherent in conventional products. The configuration of channels, conduits, or heating elements and a thermal storage medium in the therapeutic sleeve may enable even and consistent temperature distribution. The therapeutic sleeve may be integrated with a sheet, a garment, another apparatus, or it may be a standalone device for flexible use.

### BRIEF DESCRIPTION OF THE DRAWINGS

The present description may be better understood from the following detailed description read in light of the appended drawings, wherein:

FIG. 1 illustrates a cross-sectional side view of a therapeutic sleeve, according to one implementation.

FIG. 2 illustrates a top view of a therapeutic sleeve, according to one implementation.

FIG. 3 illustrates a top view of a therapeutic sleeve, according to one implementation.

FIG. 4 illustrates a perspective view of a sleeve integrated with a bedsheet, according to one implementation.

FIG. 5 illustrates a side view of a therapeutic sleeve, which includes a magnetic coupling means, according to one implementation.

FIG. 6 illustrates a perspective view of a therapeutic sleeve.

FIG. 7 illustrates a method of using a therapeutic sleeve.

FIG. 8 illustrates a perspective view of a therapeutic sleeve.

### DETAILED DESCRIPTION

A more particular description of certain implementations of a therapeutic sleeve may be had by references to the implementations shown in the drawings that form a part of this specification, in which like numerals represent like objects.

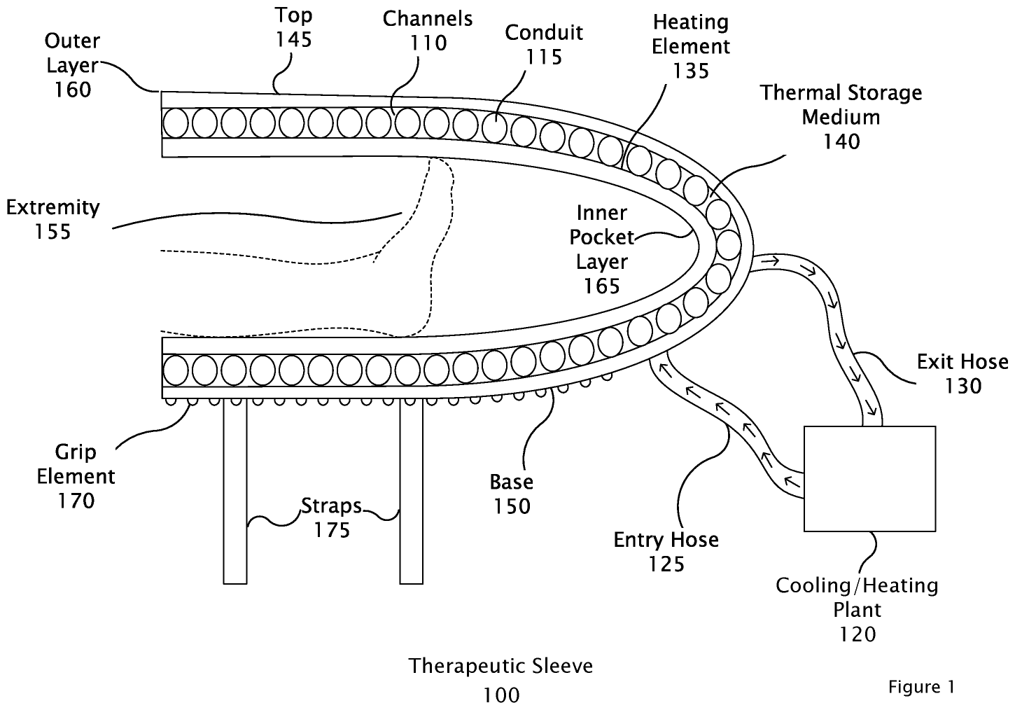

FIG. 1 illustrates a cross-sectional side view of Therapeutic Sleeve 100. In one implementation, Therapeutic Sleeve 100 may comprise a thermally insulating material having a plurality of Channels 110. Channels 110 may receive one or more Conduit 115s operable to carry a constant stream of chilled or heated working fluid throughout the sleeve. In another implementation, Therapeutic Sleeve 100 may comprise a heat-stamped bladder with channels for the working fluid to flow through.

Conduit 115 may attach to a Cooling or Heating Plant 120, which may chill or heat the working fluid to a precise temperature for a precise duration, which may be based on settings input by the user. Cooling or Heating Plant 120 may be operable to heat the working fluid, cool the working fluid, or both. Conduit 115 may couple to Entry Hose 125 and Exit Hose 130 to move the working fluid to and from the Cooling or Heating Plant. In another implementation, Channels 110 may receive a Heating Element 135, such as a coil or ribbon resistance wire, to evenly distribute heat throughout the sleeve. Thermal Storage Medium 140 may comprise a thermally conductive substance disposed around Conduit 115 or Heating Element 135 to evenly transfer heat throughout the sleeve. Thermal Storage Medium 140 may comprise a gel, silicone, water, or another substance used to facilitate a consistent application of thermal treatment while protecting the user against burn, frostbite, or skin irritation, for example.

Top 145 may fold over Base 150 of Sleeve 100 to define a pocket operable to receive a body part of the user, for example, Extremity 155. In yet another implementation, Therapeutic Sleeve 100 may comprise Base 150 and Top 145 as separate sections, which may be held together by straps, magnets, snaps, buttons, clips, adhesives, hook-and-loop fasteners, or another attachment means. Outer Layer 160 may comprise a waterproof, leakproof, and thermally insulated material, which may include foam, memory foam, or another material to reduce heat transfer between the interior of the sleeve and the surrounding environment, or to enhance comfort. Inner Pocket Layer 165 may be disposed opposite Outer Layer 160, and it may line an interior of the pocket defined by the sleeve. Inner Pocket Layer 165 or Outer Layer 160 may include fleece, cotton, nylon, a weighted material, such as a modular weighted insert as an example, or another type of material to enhance comfort. The weighted material may comprise sand, rice, beads, or another material, and may provide a soft rigidity along edges or any part of Top 145 or Base 150. The weighted material may help keep the sleeve in place, for example, to keep it from sliding off a bed. In another implementation, the bed sheet may be integrated with two sleeves that provide two temperature-controlled pockets, allowing two people in one bed to experience different temperature settings. In yet another implementation, the Therapeutic Sleeve 100 may integrate with a pillowcase, providing a temperature-controlled pillow operable to provide relief to the user's head or neck, for example. In yet another implementation, Therapeutic Sleeve 100 may be a standalone device, separate from any bedsheets or pillows, and may be held in place by magnets, for example.

Base 150 may include a Grip Element 170, for example, rubber gripping, a non-slip layer of beads, or magnets to provide friction and prevent slipping of the sleeve on a surface, such as a bed. A person skilled in the art will understand that Grip Element 170 may include other variations, including but not limited to a long, flat lead-in on the base to provide more friction while reducing a likelihood that a user's extremities may move the sleeve out of place, or a low profile, rigid support structure within the sleeve to keep the base in place.

The sleeve may include an attachment means, for example, Straps 175, which may attach at its ends to secure the sleeve to a bed or another object. A person skilled in the art will understand that a grip element or an attachment means may include other variations, including but not limited to an adjustable strap looped under a mattress to provide connection points for Velcro straps on either side of the sleeve, two “L” brackets sandwiched between a bed and a bed frame, connected to the sleeve by Velcro straps on either side of the sleeve, a magnetic puck operable to pin sheets with a broad pinch point to avoid damaging the sheet, a recessed magnet to hold corners of the sleeve in place, a low profile magnetic strip which runs the length of the sides of the sleeves and sandwiches a fitted sheet without significantly stretching the sheet, an embedded clip on the base of the sleeve which, which the base is disposed beneath a fitted sheet, creates a cavity of space operable to receive the top portion of the sleeve, and where the top includes an opposing clip to secure the top to the base.

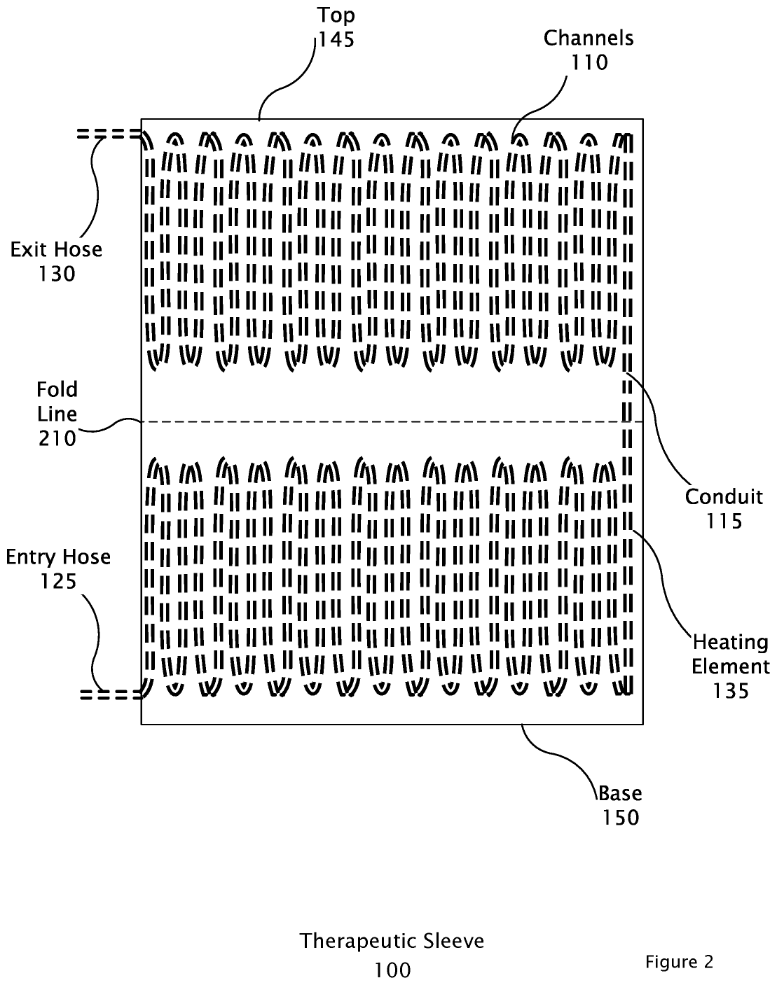

FIG. 2 illustrates a top view of Adaptive Temperature-Controlled Therapeutic Sleeve 100, according to one implementation. This example shows the sleeve lying flat in an open position. Conduit 115 may carry chilled or heated working fluid, depending on the user's desired temperature settings, or automatically generated settings, through the sleeve. Top 145 may be folded over Base 150 at a Fold Line 210. Conduit 115 may receive a steady stream of a working fluid, for example, a multiphase heat transfer fluid, a refrigerant (for example, Chlorodifluoromethane, also known as difluoromonochloromethane, hydrochlorofluorocarbon-22 (HCFC-22), or R-22, or CHClF2), steam, water, ice, or another substance. Entry Hose 125 may permit entry of the working fluid into Conduit 115. For example, when a user inputs a cooled temperature setting, Entry Hose 125 may permit entry of chilled working fluid into Conduit 115. Exit Hose 130 may permit exit of the working fluid from the sleeve. For example, when the user inputs a cooled temperature setting, Exit Hose 130 may permit exit of heated working fluid (such as previously chilled working fluid that had received a transfer of heat from the user's body). Alternatively, when the user inputs a heated temperature setting, Entry Hose 125 may permit entry of heated working fluid into Conduit 115, and Exit Hose 130 may permit exit of chilled working fluid (such as previously heated fluid which had lost heat while moving through the sleeve).

In another implementation, Channels 110 may receive two Conduit 115s. In this example, a first Conduit 115 may permit entry of working fluid into the sleeve, and a second Conduit 115 may permit exit of working fluid from the sleeve and back into Cooling/Heating Plant 120. In this example, the first Conduit 115 may attach to a first point at Cooling/Heating Plant 120, and the second Conduit 115 may attach to a second point to Cooling/Heating Plant 120. The first and second Conduit 115s may connect at or near a middle point of the sleeve. In another implementation, Channels 110 may receive a Heating Element 135, such as a coil or ribbon resistance wire, to evenly distribute heat throughout the sleeve.

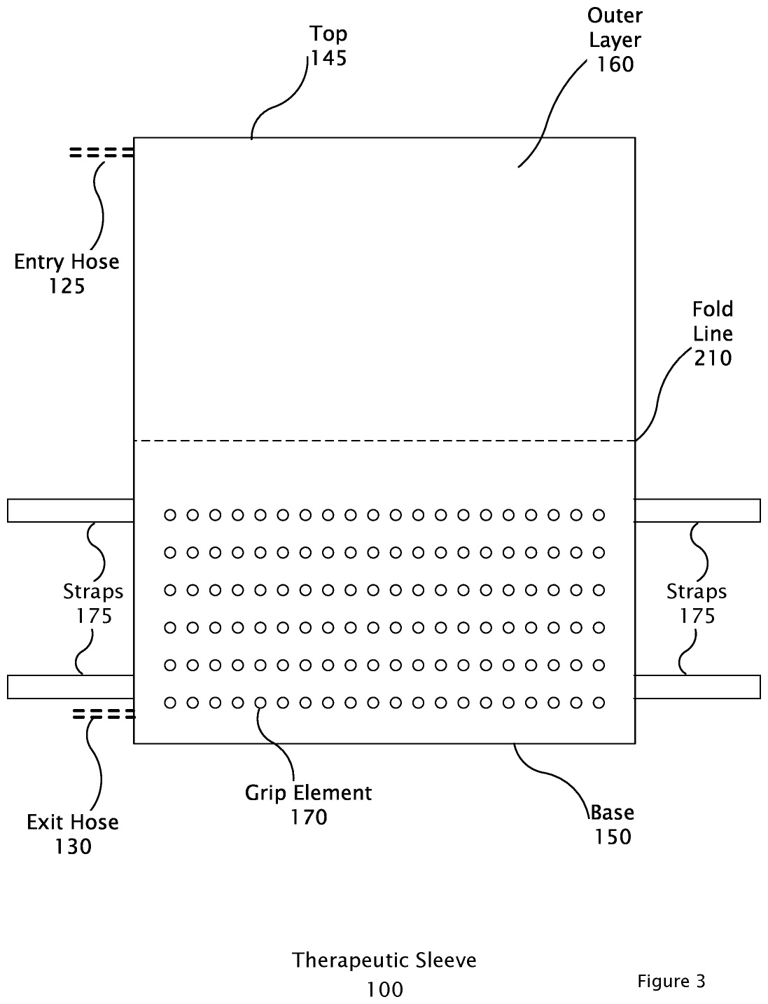

FIG. 3 illustrates a top view of Adaptive Temperature-Controlled Therapeutic Sleeve 100, according to one implementation. This example shows the sleeve lying flat in an open position. Outer Layer 160 may comprise a waterproof, leakproof, and thermally insulated material, which may include foam, memory foam, or another material to reduce heat transfer between the interior of the sleeve and the surrounding environment, or to enhance comfort. Top 145 may be disposed above Fold Line 210. Entry Hose 125 may carry working fluid into Top 145 through conduits disposed within Top 145. Exit Hose 130 may couple to Base 150 to remove working fluid from conduits disposed within Base 150. An outer surface of Base 150 may include Grip Element 170 to provide friction and prevent slipping of the sleeve on a bed or other surface. The sleeve may include Straps 175 to secure the sleeve to a bed or another object.

The adaptive temperature-controlled therapeutic sleeve may operate with a software application that enables the user to input a desired temperature and duration, set a timer, record sleep patterns or other data, or view analytics, among many possible functions, using a control panel, a remote control, or a mobile device, for example. In another implementation, temperature, duration, or other settings may be automatically generated. In yet another implementation, the sleeve may use battery-powered components, for example, a battery-powered cooling or heating plant or a battery-powered cooling or heating element, for ease of use and transportability.

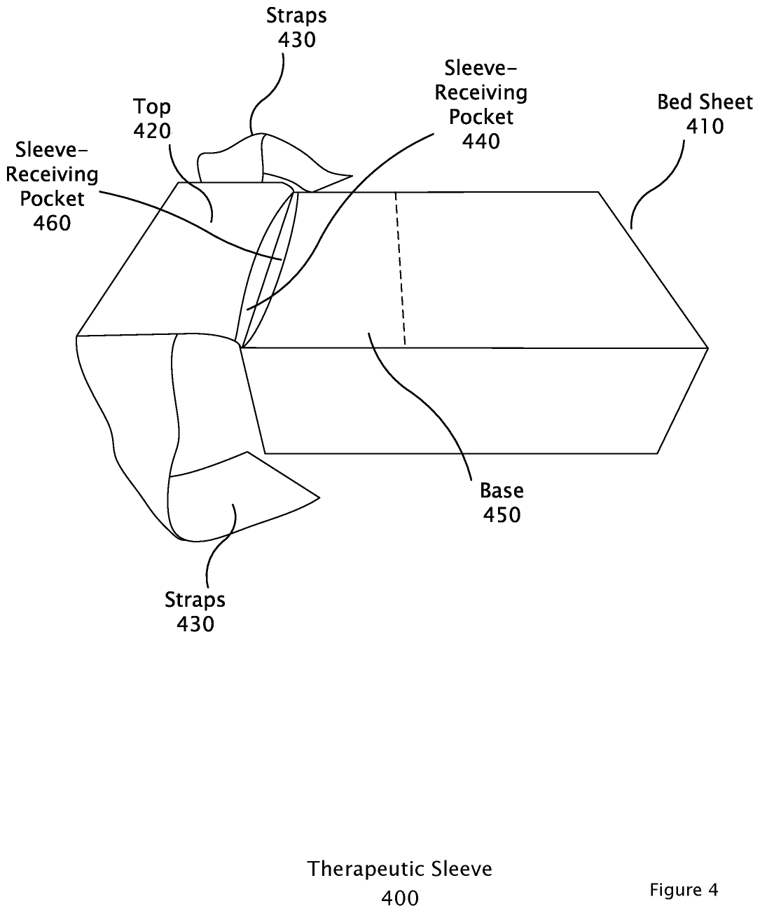

FIG. 4 illustrates a perspective view of Adaptive Temperature-Controlled Therapeutic Sleeve 400 integrated with a bedsheet, according to one implementation. This example shows the sleeve is in an opened position, integrated with Bed Sheet 410. In another implementation, the sleeve may be integrated with a garment or another apparatus configured to improve accessibility or convenience to the user. Top 420 of the sleeve may be inserted into a first Sleeve-Receiving Pocket 440 of Bed Sheet 410, and a Base 450 of the sleeve may be inserted into a second Sleeve-Receiving Pocket 460 of Bed Sheet 410. The sleeve may be integrated with a Bed Sheet 410 for any size bed (for example, a king, a California king, a queen, a twin, or a crib size model), a hospital bed, a stretcher, a gurney, a mobile unit, or the like, to readily receive the user's extremity at or near the foot of the bed. Straps 430 may secure the sleeve to the bed.

In another implementation, the bed sheet may be integrated with two sleeves that provide two temperature-controlled pockets, allowing two people in one bed to experience different temperature settings. In yet another implementation, the thermally insulated sleeve may integrate with a pillowcase, providing a temperature-controlled pillow operable to provide relief to the user's head or neck, for example.

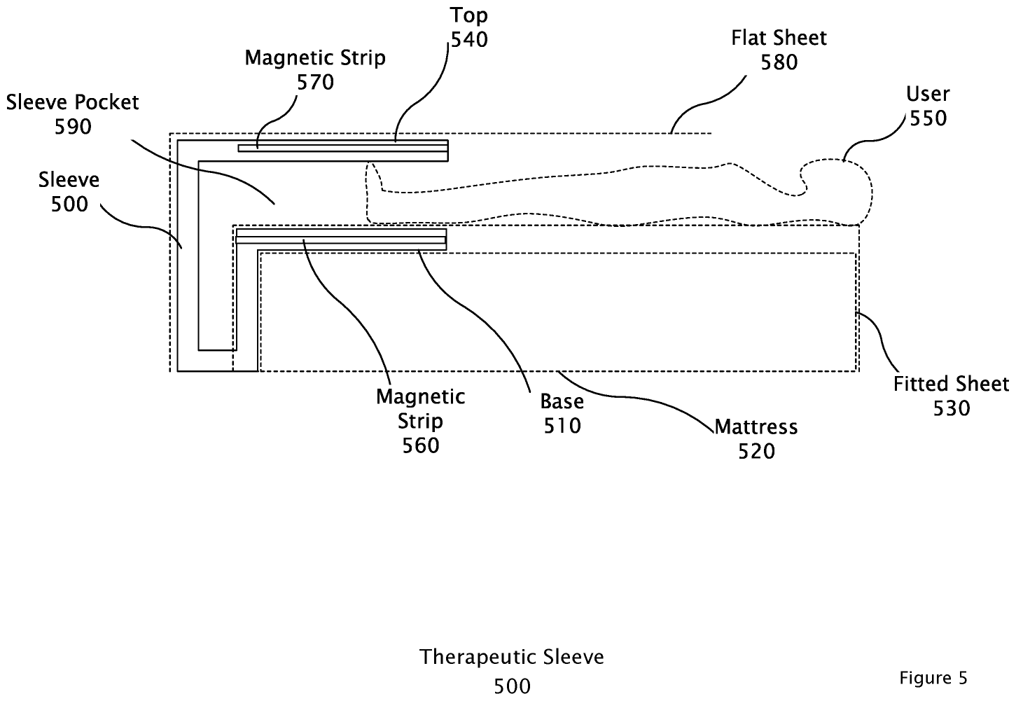

FIG. 5 illustrates a side view of an adaptive temperature-controlled therapeutic sleeve, which may include a magnetic coupling means, according to one implementation. A user may place Base 510 of Sleeve 500 onto a Mattress 520 of a bed in this example. The user may place Fitted Sheet 530 over Base 510, and then fold over Top 540 such that it is disposed above Base 510, creating Sleeve Pocket 590 to receive an extremity or another body part of User 550. A coupling means such as Magnetic Strip 560 in Base 510 may attach to a coupling means such as Magnetic Strip 570 in Top 540 to secure the Sleeve 500, which may be covered by Flat Sheet 580, while also providing pressure for comfort.

A person skilled in the art will understand that an attachment or coupling means may comprise variations including but not limited to the following. In one implementation, the attachment means may secure the top to the base through a fitted sheet. The sleeve may stay in place when a user slides extremities, such as feet, into the sleeve. The fitted sheet may not be damaged by the attachment means or other connecting features. In another implementation, two “L” brackets may get sandwiched between a bed and a bed frame and connect to the sleeve with Velcro straps on either side. In another implementation, an adjustable strap may loop under a mattress to provide connection points for Velcro straps on either side of the sleeve.

In another implementation, large magnetic pucks may pin sheets with a broad pinch point to avoid damaging fabric of a fitted sheet. Recessed magnets may hold corners in place. The magnetic pucks and magnets may be made of a ferrous metal.

In another implementation, a low-profile magnetic strip may run a length of the sleeve's sides and sandwich the fitted sheet with no stretching. In another implementation, the sleeve may have sides weighted with a heavy material such as sand, rice, or BBs, for example, which may be configured to add a soft rigidity and help keep the edges in place. In another implementation, an embedded clip on the bottom of the sleeve may allow the sheet to be folded in to help secure the sleeve in place. The top portion may have an opposing clip to secure the top to the bottom. In another implementation, the bottom portion of the sleeve may be comprised of a non-slip material. The non-slip material may include a layer with beds on its base pad to provide added friction. In another implementation, a long, flat lead-in on the bottom portion of the sleeve may provide more surface area for added friction while reducing a likelihood of pushing down the bottom portion with a user's feet. In yet another implementation, a low profile, rigid support structure may keep the bottom portion of the sleeve in place.

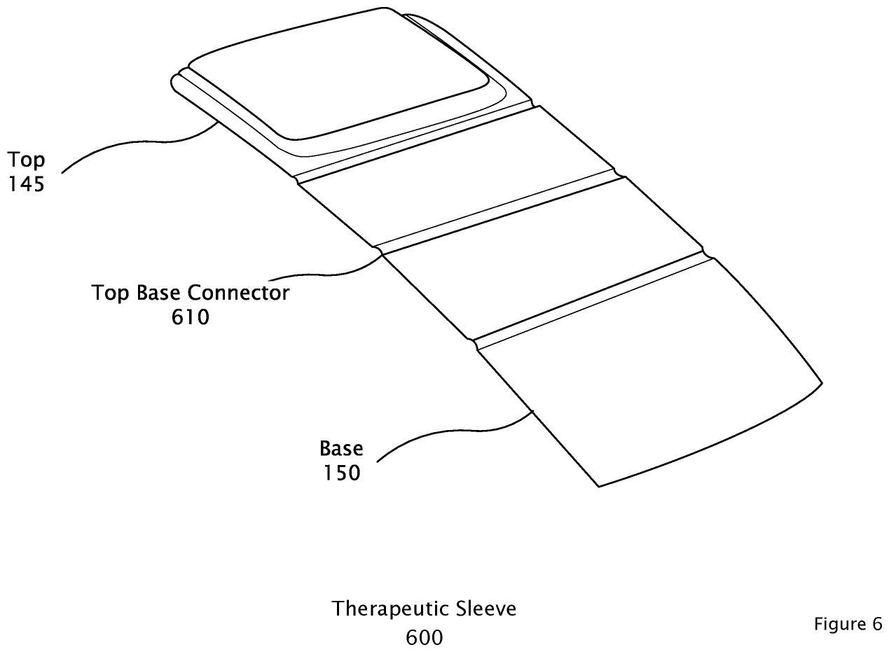

FIG. 6 illustrates a perspective view of Adaptive Temperature-Controlled Therapeutic Sleeve 600 with Top 145, Base 150, and Top Base Connector 610 attaching the top to the base.

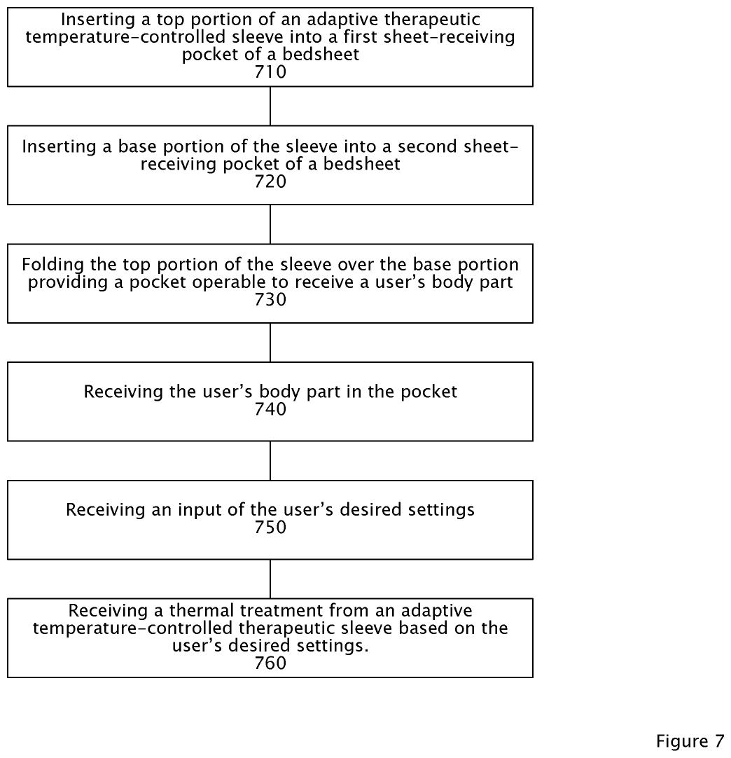

FIG. 7 illustrates a method of using an adaptive temperature-controlled therapeutic sleeve. In one implementation, the method may comprise the following steps:

Inserting a top portion of an adaptive therapeutic temperature-controlled sleeve into a first sheet-receiving pocket of a bedsheet; Inserting a base portion of the sleeve into a second sheet-receiving pocket of a bedsheet 720;

Folding the top portion of the sleeve over the base portion providing a pocket operable to receive a user's body part 730;

Receiving the user's body part in the pocket 740;

Receiving an input of the user's desired settings 750; and

Receiving a thermal treatment from an adaptive temperature-controlled therapeutic sleeve based on the user's desired settings 760.

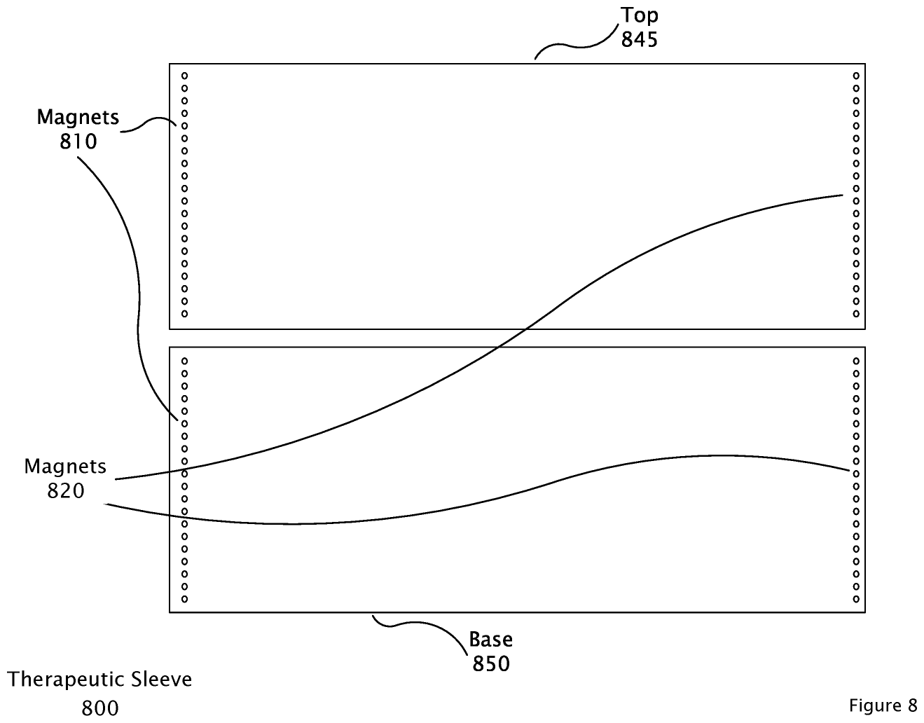

FIG. 8 illustrates a perspective view of Adaptive Temperature-Controlled Therapeutic Sleeve 800 with Top 845 and Base 850. Top 845 may be positioned over Base 850, either directly or with a bedsheet or other material separating them, such that Magnets 810 on Top 845 may attach to corresponding Magnets 820 on Base 850 to couple Top 845 to Base 850. Top 845 or Base 850 may have channels, which may receive one or more conduits operable to carry a constant stream of chilled or heated working fluid throughout the therapeutic sleeve. The conduits may attach to a cooling or heating plant, which may chill or heat the working fluid to a precise temperature for an exact duration, based on settings input by the user. In one implementation, channels may receive a heating element, such as a coil or ribbon resistance wire, to distribute heat throughout the sleeve. A thermal storage medium, for example, a gel, silicone, water, or another thermally conductive substance, may be disposed around the conduits or heating element to facilitate an even transfer of heat throughout the therapeutic sleeve.

While the detailed description above has been expressed in terms of specific examples, those skilled in the art will appreciate that many other configurations could be used. Accordingly, it will be appreciated that various equivalent modifications of the above-described implementations may be made without departing from the spirit and scope of the invention.

Additionally, the illustrated operations in the description show certain events occurring in a certain order. In alternative implementations, certain operations may be performed in a different order, modified, or removed. Moreover, steps may be added to the above-described logic and still conform to the described implementations. Further, operations described herein may occur sequentially, or certain operations may be processed in parallel.

1. A sleeve, comprising:
an outer layer, the outer layer made of a thermally insulating material;
a conduit, the conduit operable to carry a constant stream of working fluid throughout an interior of the sleeve;
a plurality of channels, the channels disposed within the interior of the sleeve, wherein the channels are operable to receive the conduit;
a cooling or heating plant, the cooling or heating plant operable to cool or heat the working fluid;
a thermal storage medium, wherein the thermal storage medium comprises a thermally-conductive substance disposed around the conduit to facilitate an even transfer of heat throughout the sleeve; and
an inner pocket layer, the inner pocket layer disposed opposite the outer layer.

2. The sleeve of claim 1, further comprising a heating element, wherein the heating element is operable to distribute heat throughout the sleeve.

3. The sleeve of claim 1, wherein a top portion of the sleeve can be folded over a base portion of the sleeve to provide a pocket operable to receive a user's body part.

4. The sleeve of claim 1, wherein the sleeve is integrated with a sheet, a garment, or an apparatus configured to improve accessibility or convenience to a user.

5. The sleeve of claim 1, further comprising an attachment means operable to couple the sleeve to a piece of furniture suitable for resting or sleeping, wherein the attachment means is selected from the list containing straps, magnets, snaps, buttons, clips, adhesives, and hook-and-loop fasteners.

6. The sleeve of claim 1, wherein the sleeve is integrated with a pillow.

7. The sleeve of claim 1, further comprising attachment means operable to couple the sleeve to a pillow, wherein the attachment means is selected from the list containing straps, magnets, snaps, buttons, clips, adhesives, and hook-and-loop fasteners.

8. The sleeve of claim 1, wherein the working fluid means is selected from the list containing a multiphase heat transfer fluid, a refrigerant, steam, water, and ice.

9. The sleeve of claim 1, wherein the thermal storage medium is selected from the list consisting of a gel, silicone, or water.

10. The sleeve of claim 1, wherein the outer layer includes a material to reduce heat transfer between the interior of the sleeve and a surrounding environment, or to enhance comfort, wherein the material is selected from the list containing foam or memory foam.

11. The sleeve of claim 3, wherein a gripping material is disposed on the base of the sleeve.

12. The sleeve of claim 1, wherein the cooling or heating plant is battery-powered.

13. The sleeve of claim 1, further comprising a remote control.

14. The sleeve of claim 1, further configured to be operated using a mobile device.

15. A method, comprising:
inserting a top portion of a temperature-controlled sleeve into a first sheet-receiving pocket of a bedsheet;
inserting a base portion of the sleeve into a second sheet-receiving pocket of the bedsheet;
folding the top portion of the sleeve over the base portion providing a pocket operable to receive a user's body part;
receiving the user's body part in the pocket;
receiving an input of the user's desired settings; and
receiving a thermal treatment from an adaptive temperature-controlled therapeutic sleeve based on the user's desired settings.
